(19) United States
(12) Patent Application Publication
(10) Pub. No.: US 2025/0134995 A1
(43) Pub. Date: May 1, 2025

(54) SAPONIN-BASED ADJUVANTS AND METHODS OF CHARACTERIZING SAME

(71) Applicants: Anna-Karin E. PALM, Uppsala (SE); Cecilia CARNROT, Uppsala (SE); Linda STERTMAN, Uppsala (SE)

(72) Inventors: Anna-Karin E. PALM, Uppsala (SE); Cecilia CARNROT, Uppsala (SE); Linda STERTMAN, Uppsala (SE)

(21) Appl. No.: 18/927,547

(22) Filed: Oct. 25, 2024

Related U.S. Application Data
(60) Provisional application No. 63/592,974, filed on Oct. 25, 2023.
(60) Provisional application No. 63/560,475, filed on Mar. 1, 2024.

(51) Int. Cl. A61K 39/39; A61K 39/00

(52) U.S. Cl. CPC A61K 39/39; A61K 2039/54; A61K 2039/55555; A61K 2039/55577

(57) ABSTRACT

Immunogenic compositions that include an adjuvant formed from nanoparticles of saponins combined with cholesterol and phospholipids have particular interactions with body tissue and immune cells. These interactions include increased immune response in the draining lymph node, strong activation of T and B cells in the lymph node, and rapid clearance of the saponin fraction while the cholesterol fraction remains in the system.

### RELATED APPLICATIONS

The present invention claims priority to U.S. Provisional Patent Application No. 63/592,974, filed on Oct. 25, 2023 and U.S. Provisional Patent Application No. 63/560,475, filed on Mar. 1, 2024, each of which is incorporated herein in its entirety by reference.

### FIELD OF THE DISCLOSURE

The present invention relates generally to adjuvants for use with vaccines, particularly saponin-based vaccines, and methods for characterizing them.

### BACKGROUND

Vaccines are among the most successful and cost-effective ways to battle infectious diseases. Importantly, vaccines are also a crucial part of pandemic preparedness as novel pathogens emerge, such as the SARS-COV-2 virus; the cause of the COVID-19 pandemic. Such vaccines must create a strong enough stimulus to drive the desired immune response and be clinically effective, while retaining acceptable tolerability and safety for widespread uptake. One promising approach is recombinant subunit vaccines, in which the use of highly purified, pathogen-specific recombinant proteins enables specific targeting of the immune response to relevant epitopes by robust and thermally stable formulations. However, such recombinant proteins are generally poorly immunogenic and need to be paired with an adjuvant to induce robust antibody responses. Vaccine adjuvants are compounds that can be used in vaccines to increase the magnitude and/or tailor the quality of the immune response to a particular antigen. Matrix-M™ adjuvant is a novel adjuvant, which is a critical component of the NVX-CoV2373 vaccine that recently received authorization for use in humans by multiple regulatory agencies (among those the U.S. Food and Drug Administration and the European Medicines Agency) and is listed on the World Health Organization's emergency use listing for COVID-19 vaccines. Matrix-M™ adjuvant is also included in several other candidate vaccines in the clinical developmental phase, including targeting seasonal influenza (alone and in combination with COVID-19), malaria, and Ebola Virus Disease.

Matrix-M™ adjuvant is a saponin-based adjuvant, made with saponins from the Chilean soap bark tree (Quillaja Saponaria Molina) that are formulated with cholesterol and phospholipids into cage-like nanoparticles. Two types of such nanoparticles, Matrix-ATM and Matrix-C™, each made up with a specific saponin fraction (Fraction-A; Fr-A, and Fraction-C; Fr-C), are mixed at a set ratio to form Matrix-M™ adjuvant. The main component is Matrix-ATM which comprises 85% of Matrix-M™, with Matrix-C™ comprising the remaining 15%.

Matrix-M™ adjuvant has antigen dose-sparing properties and promotes the induction of a balanced Th1/Th2 type CD4+ T cell immune response, driving the generation of IgG subclasses with different effector functions. Importantly, vaccines adjuvanted with Matrix-M™ have been shown to broaden the antibody response to include cross-protective antibodies. In addition, Matrix-M™ adjuvantation of purified protein antigens can also induce antigen specific CD8+ T cell responses. Importantly, several large clinical trials have shown that vaccines adjuvanted with Matrix-M™ have an acceptable safety profile.

To date there is a limited understanding of the pharmacokinetics and biodistribution of saponin-based adjuvants in general and of Matrix-M™ adjuvant in particular. In addition to addressing these issues, results from biodistribution studies can help to further define the mechanism of action (MoA) and safety profile of Matrix-M™-adjuvanted vaccines. Thus, it is important to understand the biodistribution of Matrix-M™.

### SUMMARY

The present invention relates generally to saponin-based adjuvants and their effects on tissues and organs.

One embodiment of the invention is directed to a method of providing a vaccination composition to a mammalian subject that includes injecting the vaccination composition at an intramuscular injection site of the subject, the vaccination composition comprising a saponin-based adjuvant and the intramuscular injection site being associated with a draining lymph node (dLN). Following injection of the vaccination composition at the intramuscular injection site, a fraction of T cells in the dLN that are positive for i) CD45 and ii) the saponin-based adjuvant has a minimum value at a time greater than one hour following injection of the vaccination composition at the intramuscular injection site and less than 24 hours following injection of the vaccination composition at the intramuscular injection site, the fraction of T cells in the dLN that are positive for i) CD45 and ii) the saponin-based adjuvant at a time 24 hours following injection of the vaccination composition having a value greater than the minimum value.

Another embodiment of the invention is directed to a method of providing a vaccination composition to a mammalian subject that includes injecting the vaccination composition at an intramuscular injection site of the subject, the intramuscular injection site being associated with a draining lymph node (dLN). The vaccination composition includes a saponin-based adjuvant. Over a time period between about one hour and about twenty four hours following injection of the vaccination composition, a fraction of B cells in the dLN that are positive for i) CD45 and ii) the adjuvant has a maximum value at a time less than three hours following injection of the mixture at the intramuscular injection site.

Another embodiment of the invention is directed to a method for administering a vaccination composition to a mammalian subject that includes injecting the vaccination composition at an intramuscular injection site of the subject, the intramuscular injection site being associated with a draining lymph node (dLN). The vaccination composition comprises an antigen and a saponin-based adjuvant, the saponin-based adjuvant comprising a saponin component and a cholesterol component. Following injection of the vaccination composition at the intramuscular injection site of the subject, concentration of the saponin component in the subject's blood plasma peaks at a time less than 3 hours after the injection, and concentration of the cholesterol component in the subject's blood plasma peaks at a time greater than 6 hours after the injection.

Another embodiment of the invention is directed to a method for administering a vaccination composition to a mammalian subject that includes injecting the vaccination composition at an intramuscular injection site of the subject, the intramuscular injection site being associated with a draining lymph node (dLN). The vaccination composition comprises an antigen and a saponin-based adjuvant. The saponin-based adjuvant includes a saponin component and a cholesterol component. Following injection of the vaccination composition at the intramuscular injection site of the subject, concentration of the saponin component in one of the subject's intestines, lungs and heart remains approximately zero up to 168 hours after the injection, and concentration of the cholesterol component in the one of the subject's intestines, lungs and heart peaks at a time greater than 24 hours after the injection.

Another embodiment of the invention is directed to a method for administering a vaccination composition to a mammalian subject that includes injecting the vaccination composition at an intramuscular injection site of the subject, the intramuscular injection site being associated with a draining lymph node (dLN). A cytokine response induced by the vaccine composition at the dLN measured 24 hours after injection of the mixture is stronger than a cytokine response at the dLN measured 6 hours after injection of the mixture.

### BRIEF DESCRIPTION OF THE DRAWINGS

The invention may be more completely understood in consideration of the following detailed description of various embodiments of the invention in connection with the accompanying drawings, in which:

FIG. 1 schematically illustrates representative structures of Fraction-A and altered Fraction-A, in which the aldehyde in position C-23 on the triterpene core was reduced to a hydroxyl group containing tritium.

FIGS. 2A and 2B: mice were immunized subcutaneously with 1 μg rS and 5 μg Matrix-M™, with 1 μg rS and 5 μg Matrix-M™ 50/50 (formulated from 50% altered and 50% unaltered Fr-A material (Matrix-M™ 50/50)), or with 1 μg SARS-COV-2rS alone (“Ag only”) on Day 0 and Day 21. Serum samples obtained 20 days (FIG. 3A) and 28 days (FIG. 3B) after the primary immunization were evaluated for IgG1 and IgG2a antibody titers against rS protein and for hACE2 receptor-inhibiting antibody titers by ELISA. Individual titers are shown for each symbol, horizontal bars represent geometric mean titers and error bars represent 95% confidence intervals (CI). The data were analyzed by one-way ANOVA with Tukey's multiple comparisons test. Statistically significant differences between groups are denoted with * (p<0.05), ** (p<0.01); **** (p<0.0001). The number of mice tested was 10 (n=10).

FIG. 3 presents the study design for the biodistribution study, describing the group composition and timepoints for measurements of radioactivity by liquid scintillation counting.

FIGS. 4A-4D show the distribution of saponins and cholesterol at the injection site and to local lymph nodes. Matrix-M™ adjuvant was formulated with either radiolabeled 3H-saponin or 3H-cholesterol, the former administered either with or without SARS-COV-2 rS antigen. The activity was measured in the quadriceps femoris muscle, FIG. 4A; iliac lymph nodes, FIG. 4B; inguinal lymph nodes, FIG. 4C; and popliteal lymph nodes, FIG. 4D, pooled left and right, using liquid scintillation and expressed as % of injected dose (ID) per gram of tissue. In each graph, there is a cluster of three columns of data points for each timepoint. The central column of darker points denotes data obtained with radiolabeled 3H-saponin with SARS-COV-2 rS antigen (labeled in FIG. 4C as “Matrix-M (3H-Sap+SARS-COV-2 rS)”). The column of lighter points to the left of the central column represents data obtained using radiolabeled saponin alone (labeled in FIG. 4C as “Matrix-M (3H-Sap)”). The column of lighter points to the right of the central column represents data obtained using radiolabeled cholesterol (labeled in FIG. 4C as “Matrix-M (3H-Chol)”). Activity (DPM) below the LOQ (703 DPM) was set to 703 DPM and the % ID/g tissue was determined accordingly. Each datapoint represents an individual animal, the horizontal bar denotes the mean+/−standard deviation. Datapoints below the LOQ are shown as X. The data were analyzed separately for each tissue and timepoint by two-way ANOVA with Tukey's multiple comparisons test. Statistically significant differences between groups are denoted with *, p<0.05; **, p<0.01; ***, p<0.001; ****, p<0.0001. n=5-6.

FIGS. 5A-5J show the distribution of saponins and cholesterol to various sites around the body. Matrix-M™ adjuvant was formulated with either radiolabeled 3H-saponin or 3H-cholesterol, the former administered with or without SARS-COV-2 rS antigen. The activity was measured by liquid scintillation and expressed as % of injected dose (ID) per mL in plasma, FIG. 5A; urine, FIG. 5B; kidney, FIG. 5C; liver, FIG. 5D; intestines, FIG. 5E; spleen, FIG. 5F; lungs, FIG. 5G; bone marrow, FIG. 5H; heart, FIG. 5I; and brain, FIG. 5J. The columns of datapoints at each timepoint represent data gathered in the same way as described above for FIGS. 4A-4D, i.e. the central column of darker datapoints represents data obtained from radiolabeled saponin administered with SARS-COV-2 rS antigen, the column of lighter datapoints to the left of the central column represents data obtained from radiolabeled saponin alone, and the column of lighter datapoints to the right of the central column represents data obtained using radiolabeled cholesterol, as shown for FIG. 5I. Activity (DPM) below the LOQ (703 DPM) was set to 703 DPM and the % ID/g tissue was determined accordingly. Each datapoint represents an individual animal, the horizontal bar denotes the mean +/−standard deviation. Datapoints below the LOQ are shown as X. The data were analyzed separately for each tissue and timepoint by two-way ANOVA with Tukey's multiple comparisons test. Statistically significant differences between groups are denoted with *, p<0.05; **, p<0.01; ***, p<0.001; ****, p<0.0001. n=4-6.

FIGS. 6A-6C show the distribution of saponins and cholesterol to reproductive organs. Matrix-M™ adjuvant was formulated with either radiolabeled 3H-saponin or 3H-cholesterol, the former administered with or without SARS-COV-2 rS antigen. The activity was measured in testes (males), FIG. 6A; ovaries, FIG. 6B; and uterus, FIG. 6C, by liquid scintillation and expressed as % of injected dose (ID) per gram tissue. The columns of datapoints at each timepoint represent data gathered in the same way as described above for FIGS. 4A-4D, i.e. the central column of darker datapoints represents data obtained from radiolabeled saponin administered with SARS-CoV-2 rS antigen, the column of lighter datapoints to the left of the central column represents data obtained from radiolabeled saponin alone, and the column of lighter datapoints to the right of the central column represents data obtained using radiolabeled cholesterol, as shown for FIG. 6A. Activity (DPM) below the LOQ (703 DPM) was set to 703 DPM and the % ID/g tissue was determined accordingly. Each datapoint represents an individual animal, the horizontal bar denotes the mean +/−standard deviation. Datapoints below the LOQ are shown as X. The data were analyzed separately for each tissue and timepoint by two-way ANOVA with Tukey's multiple comparisons test. Statistically significant differences between groups are denoted with *, p<0.05; **, p<0.01; ***, p<0.001; ****, p<0.0001. n=2-3.

FIG. 7 schematically illustrates the reaction to produce fluorescently-modified Fraction-A from Fraction-A.

FIGS. 8A and 8B present results from flow cytometry analysis using fluorescently-labeled saponins, for different cell types. The x-axis shows, for ten different cell types, the percentage of that cell type that is both CD45+ and is attached to a fluorescently-labeled Matrix-ATM at different times, 0.5 h, 1 h, 3 h, 6 h, 18 h, and 24 h. The cell types from left to right are cells, natural killer T (NKT) cells, B cells and others. The y-axis shows the percentage of a type of cell that is positive for CD45 and the fluorescently labeled Matrix-ATM. FIG. 8A shows measurements taken at the intramuscular injection site. FIG. 8B shows measurements taken at the dLN.

FIGS. 9A and 9B present heat maps showing the occurrence of various cytokines present at the muscle injection site (FIG. 9A) and dLN (FIG. 9B) at 6 h, 24 h, 48 h, 72 h, and 168 h after injection for various adjuvants with NIV antigen. In each case the mouse was injected with antigen with Matrix-M, antigen with AS01a, antigen with alhydrogel, or antigen alone without adjuvant. The cytokines tested for were CXCL10, IL-1b, TNF-α, IL-30, IL-6, CXCL1, CCL2, IL-5, CXCL2, CCL3, IL-33, and IFN-g.

While the invention is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the invention as defined by the appended claims.

### DETAILED DESCRIPTION

### Definitions

As used herein, and in the appended claims, the singular forms “a”, “an”, and “the” include plural referents unless the context clearly dictates otherwise. Thus, for example, reference to “a protein” can refer to one protein or to mixtures of such protein, and reference to “the method” includes reference to equivalent steps and/or methods known to those skilled in the art, and so forth.

As used herein, the term “adjuvant” refers to a compound that, when used in combination with an immunogen, augments or otherwise alters or modifies the immune response induced against the immunogen. Modification of the immune response may include intensification or broadening the specificity of either or both antibody and cellular immune responses.

As used herein, the term “about” or “approximately” when preceding a numerical value indicates the value plus or minus a range of 10%. For example, “about 100” encompasses 90 and 110.

As used herein, “substantially” refers to isolation of a substance (e.g. a compound, polynucleotide, or polypeptide) such that the substance forms the majority percent of the sample in which it is contained. For example, in a sample, a substantially purified component comprises 85%, preferably 85%-90%, more preferably at least 95%-99.5%, and most preferably at least 99% of the sample. If a component is substantially replaced the amount remaining in a sample is less than or equal to about 0.5% to about 10%, preferably less than about 0.5% to about 1.0%.

The terms “treat,” “treatment,” and “treating,” as used herein, refer to an approach for obtaining beneficial or desired results, for example, clinical results. For the purposes of this disclosure, beneficial or desired results may include inhibiting or suppressing the initiation or progression of an infection or a disease; ameliorating, or reducing the development of, symptoms of an infection or disease; or a combination thereof.

“Prevention,” as used herein, is used interchangeably with “prophylaxis” and can mean complete prevention of an infection or disease, or prevention of the development of symptoms of that infection or disease; a delay in the onset of an infection or disease or its symptoms; or a decrease in the severity of a subsequently developed infection or disease or its symptoms.

As used herein an “effective dose” or “effective amount” refers to an amount of an antibody sufficient to induce an immune response that reduces at least one symptom of pathogen infection. An effective dose or effective amount may be determined e.g., by measuring amounts of neutralizing secretory and/or serum antibodies, e.g., by plaque neutralization, complement fixation, enzyme-linked immunosorbent (ELISA), or microneutralization assay.

### Matrix-A™ Adjuvant

An embodiment of the present invention is generally directed to the biodistribution of radiolabeled saponin (Fr-A) or cholesterol incorporated into the Matrix-A™ particles, which make up 85% of the Matrix-M™ adjuvant. By a head-to-head comparison of Matrix-A™ particles made up with radiolabeled Fr-A or with radiolabeled cholesterol, the biodistribution patterns of these two critical components was discerned separately. In addition, the presence of the rS nanoparticle antigen included in the NVX-CoV2373 vaccine is found not to affect the biodistribution and/or excretion of the saponins.

Vaccine adjuvants are substances that enhance and modulate the immunogenicity of antigens. Due to their capacity to activate components of the innate immune system, adjuvants increase the magnitude, broaden the specificity, change the humoral and cellular characteristics of the vaccine-induced immune responses, and improve memory responses. These properties have enabled the development of vaccines for target populations (e.g., infants, older adults, and immunocompromised persons) and infectious diseases (e.g., malaria, herpes zoster, and avian influenza) for which traditional vaccines have been unsuccessful or of limited efficacy. In addition, adjuvants may also help reduce the number of doses needed to immunize an individual or reduce the amount of antigen needed in each dose (dose-sparing effect).

The need for new adjuvants has been strongly driven by the development of vaccines containing highly purified antigens derived from recombinant technologies, which are more precisely characterized and safer, but potentially less immunogenic, than live-attenuated or inactivated whole-cell vaccines. Indeed, most older live-attenuated, inactivated, or toxoid vaccines are “self-adjuvanted” because they contain intrinsic molecules called pathogen-associated molecular patterns that are recognized by cells of the innate immune system such as monocytes, macrophages, and dendritic cells (DCs) via a range of so-called pattern recognition receptors. In contrast, purified recombinant protein vaccines often lack these microbial patterns necessary to trigger innate immune responses. Adding adjuvants to the vaccine antigens triggers a rapid innate immune response at the injection site and/or the draining lymph nodes (dLNs) by attracting and activating antigen-presenting cells (APCs). The APCs can then process and present the antigen to CD4+ and CD8+ T cells. Both T-cell types are capable of providing cellular effector functions and CD4+ T cells help to develop a mature adaptive immune response. This acute response sets the stage to create immunological memory that can persist for years and react more rapidly upon subsequent infection. By modifying the initial signal provided to the innate immune system, the adjuvant also influences the type of adaptive immune responses induced against the vaccine antigen. Although adjuvants have been used for more than a century, the mechanisms of action of the ones most widely used in humans-aluminum salts, oil-in-water emulsions, saponin-based adjuvants (SBAs), and Toll-like receptor (TLR) agonists—are still not fully understood, and animal models may not fully reflect the more nuanced picture found in human data.

Saponins are a large family of glycoconjugates that share a triterpene structure with a variety of glycoside side chains. Most saponins used in vaccine adjuvants and food are extracted through a sustainable process from the bark of the South American soapbark tree Quillaja saponaria Molina. These bark extracts contain a heterogeneous mixture of dozens of closely related saponins with structurally different glycosylation or acylation patterns that also affect their biological activities. Of the numerous components that can be purified, Q. saponaria Molina QS-21 is the best characterized, both structurally and functionally, and is associated with a potent adjuvant activity. However, QS-21 is chemically unstable in alkaline conditions, shows hemolytic activity in vitro, and is associated with immediate pain at the injection site. These limitations of free QS-21 can be attenuated by incorporating it into particles with cholesterol, as done in the liposome-based Adjuvant System 01 (AS01) and the Army Liposome Formulation Q (ALFQ), or with a combination of cholesterol and phospholipid, as in immune-stimulating complexes (ISCOMs) or Matrix-M nanoparticles. In addition to “quenching” saponin hemolytic activity, the formulation into nanoparticles allows targeting the delivery of the adjuvant to phagocytic cells, thus focusing stimulation to appropriate cells. Saponin adjuvants can also synergize with other classes of adjuvants, such as TLR agonists. This characteristic is used in the liposome-based adjuvant AS01, which combines QS-21 and the TLR4 agonist MPL (3-O-desacyl-4′-monophosphoryl lipid A), a nontoxic derivative from Salmonella minnesota lipopolysaccharide. The QS-21 and MPL components of this adjuvant enhance antigen-specific antibody responses and promote T-cell responses. AS01 is currently used in the licensed vaccines against malaria and herpes zoster, both developed by GSK (Rixensart, Belgium). The synergistic adjuvant effects of saponins and MPL are also seen when saponins and MPL are incorporated into ISCOM nanoparticles.

ISCOMs were initially developed to improve the immunogenicity of membrane-derived viral glycoproteins by coformulation of these glycoproteins with adjuvant-active Quillaja saponins present in the bark extract fractions. The characteristic structure of ISCOMs relies on the strong affinity between saponins and cholesterol. ISCOMs are stable particles formed by Q. saponaria saponins, cholesterol, and phospholipids, in which multiple copies of antigens are physically incorporated into a matrix of saponins and lipids. ISCOMs are 40-nm-diameter particles with typical rigid cage-like structures. ISCOMs induce strong and long-lasting antigen-specific humoral and cellular immune responses, including CD4+ helper T cells and CD8+ cytotoxic T cells. However, a limitation of the ISCOM system is that only hydrophobic membrane proteins are readily incorporated into ISCOMs without modification. Manufacturing challenges that arose when incorporating a broader variety of antigens into the ISCOM particles led to the discovery that similar characteristic structures were produced even without antigen incorporation. These complexes, later called Matrix, constituted a potent adjuvant that could be simply mixed with a broad range of antigens. Although antigens are not physically linked to the particles, these formulations retain the ISCOMs' ability to induce strong humoral and cellular immune responses and, as they are not limited to hydrophobic membrane proteins, they potentially have broader applications.

The technology was further developed to produce the Matrix-M formulation. The Matrix-M adjuvant consists of two different populations of physically stable nanoparticles mixed at a defined ratio (85% Matrix-A+15% Matrix-C). Matrix-A™ and Matrix-C™ contain different Q. saponaria saponin fractions with complementary properties. Matrix-C particles contain Fraction-C saponins (mainly consisting of QS-21), which have strong adjuvant activity but are reactogenic in mice, as measured by lethargy and lethality. Matrix-A particles contain Fraction-A saponins, which have a weaker adjuvant activity than Fraction-C at the same doses but are better tolerated in mice. The nanoparticles contain cholesterol and phospholipids. A combination of these two types of particles was tested and reduced the reactogenicity observed in animal models while preserving the adjuvant activity. In addition, Matrix-M is stable in aqueous solution at 2-8° C. for several years. Matrix-M is the adjuvant used in NVX2373 SARS-COV-2 recombinant spike protein-based vaccine (Novavax) as well as several vaccines that are currently being, or have been, evaluated in clinical trials. These include COVID-19, seasonal influenza, COVID-19+seasonal influenza, pandemic (H5N1) influenza, avian (H7N9) influenza, respiratory syncytial virus (RSV), malaria, ebola, genital herpes simplex virus (HSV), rabies and Epstein-Barr virus.

### Formulations, Administration and Dosing

Pharmaceutical formulations of the invention contain Matrix-M™ adjuvant in addition to an antigen. The formulations may contain naked antibody, immunoconjugate, or fusion protein as the antigen in an amount effective for producing the desired response in a unit of weight or volume suitable for administration to a human patient, and are preferably sterile.

A composition may be formulated with a pharmaceutically acceptable carrier. The term “pharmaceutically acceptable” means one or more non-toxic materials that do not interfere with the effectiveness of the biological activity of the active ingredients. Such preparations may routinely contain salts, buffering agents, preservatives, compatible carriers, and optionally other therapeutic agents. Such pharmaceutically acceptable preparations may also routinely contain compatible solid or liquid fillers, diluents or encapsulating substances which are suitable for administration into a human. When used in medicine, the salts should be pharmaceutically acceptable, but non-pharmaceutically acceptable salts may conveniently be used to prepare pharmaceutically acceptable salts thereof and are not excluded from the scope of the invention. Such pharmacologically and pharmaceutically acceptable salts include, but are not limited to, those prepared from the following acids: hydrochloric, hydrobromic, sulfuric, nitric, phosphoric, maleic, acetic, salicylic, citric, boric, formic, malonic, succinic, and the like. Also, pharmaceutically acceptable salts can be prepared as alkaline metal or alkaline earth salts, such as sodium, potassium or calcium salts. The term “carrier” denotes an organic or inorganic ingredient, natural or synthetic, with which the active ingredient is combined to facilitate the application. The components of the pharmaceutical compositions also are capable of being co-mingled with the antibodies of the present invention, and with each other, in a manner such that there is no interaction which would substantially impair the desired pharmaceutical efficacy.

According to certain aspects of the invention, compositions can be prepared for storage by mixing the antigen having the desired degree of purity with optional physiologically acceptable carriers, excipients or stabilizers (Remington's Pharmaceutical Sciences, 16th edition, Osol, A. Ed. (1999)), in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptide; proteins, such as serum albumin, gelatin, or immunoglobulins; hydrophilic polymers such as polyvinylpyrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN, PLURONICS™ or polyethylene glycol (PEG).

Compositions also may contain, optionally, suitable preservatives, such as: benzalkonium chloride; chlorobutanol; parabens and thimerosal.

Compositions suitable for parenteral administration conveniently comprise a sterile aqueous or non-aqueous preparation, which is preferably isotonic with the blood of the recipient. This preparation may be formulated according to known methods using suitable dispersing or wetting agents and suspending agents. The sterile injectable preparation also may be a sterile injectable solution or suspension in a non-toxic parenterally acceptable diluent or solvent, for example, as a solution in 1,3-butanediol. Among the acceptable vehicles and solvents that may be employed are water, Ringer's solution, and isotonic sodium chloride solution. In addition, sterile, fixed oils are conventionally employed as a solvent or suspending medium. For this purpose any bland fixed oil may be employed including synthetic mono- or di-glycerides. In addition, fatty acids such as oleic acid may be used in the preparation of injectables. Carrier formulation suitable for oral, subcutaneous, intravenous, intramuscular, etc. administration can be found in Remington's Pharmaceutical Sciences, Mack Publishing Co., Easton, PA.

The active ingredients may also be entrapped in microcapsule prepared, for example, by coacervation techniques or by interfacial polymerization, for example, hydroxymethylcellulose or gelatin-microcapsule and poly-(methylmethacylate) microcapsule, respectively, in colloidal drug delivery systems (for example, liposomes, albumin microspheres, microemulsions, nanoparticles and nanocapsules) or in macroemulsions. Such techniques are disclosed in Remington's Pharmaceutical Sciences 16th edition, Osol, A. Ed. (1980).

The formulations to be used for in vivo administration are typically sterile. This is readily accomplished by filtration through sterile filtration membranes.

In certain embodiments, a pharmaceutical composition of the invention is stable at 4° C. In certain embodiments, a pharmaceutical composition of the invention is stable at room temperature.

Administration of compositions of the invention to a human patient can be by any route, including but not limited to intravenous, intradermal, transdermal, subcutaneous, intramuscular, inhalation (e.g., via an aerosol), buccal (e.g., sub-lingual), topical (i.e., both skin and mucosal surfaces, including airway surfaces), intrathecal, intraarticular, intraplural, intracerebral, intra-arterial, intraperitoneal, oral, intralymphatic, intranasal, rectal or vaginal administration, by perfusion through a regional catheter, or by direct intralesional injection. In one embodiment, compositions of the invention are administered by intravenous push or intravenous infusion given over defined period (e.g., 0.5 to 2 hours). Compositions of the invention can be delivered by peristaltic means or in the form of a depot, although the most suitable route in any given case will depend, as is well known in the art, on such factors as the species, age, gender and overall condition of the subject, the nature and severity of the condition being treated and/or on the nature of the particular composition (i.e., dosage, formulation) that is being administered.

In embodiments, the dose of a composition comprising an antigen is measured in units of mg (antigen)/kg of patient body weight. In other embodiments, the dose of a composition comprising the antigen is measured in units of mg/kg of patient lean body weight (i.e., body weight minus body fat content). In yet other embodiments, the dose of a composition comprising antigen is measured in units of mg/m² of patient body surface area. In yet other embodiments, the dose of a composition comprising antigen is measured in units of mg per dose administered to a patient. Any measurement of dose can be used in conjunction with compositions and methods of the invention and dosage units can be converted by means standard in the art.

In embodiments, the amount of adjuvant present in the composition may range from about 1 μg to 500 μg, in other embodiments from about 25 μg to about 100 μg. In embodiments, the composition may contain 25 μg, 30 μg, 35 μg, 40 μg, 45 μg, 50 μg, 55 μg, 60 μg, 65 μg, 70 μg, or 75 μg of adjuvant.

Those skilled in the art will appreciate that dosages can be selected based on a number of factors including the age, sex, species and condition of the subject. For example, effective amounts of compositions of the invention may be extrapolated from dose-response curves derived in vitro test systems or from animal model (e.g., the cotton rat or monkey) test systems. Models and methods for evaluation of the effects of antibodies are known in the art (Wooldridge et al., Blood, 89 (8): 2994-2998 (1997)), incorporated by reference herein in its entirety).

Examples of dosing regimens that can be used in methods of the invention include, but are not limited to, daily, three times weekly (intermittent), weekly, every 14 days, every month, every 6-8 weeks, every 2 months, every 6 months, or every year. Other dosing regimens may include a single dose, or may include a second dose administered sometime after the initial dose, for example spaced from the initial dose by two weeks, one month, six months, or a year.

### Experimental Methods

Female BALB/c mice (8-10 weeks old) and female/male CD-1 IGS mice (8 weeks old) were obtained from Charles River Laboratories (CRL, Germany). At termination, animals were either euthanized by cervical dislocation (SVA) or by deep anesthesia with pentobarbital (180 mg/kg, intraperitoneal).

Matrix-M™ adjuvant is composed of two 40 nm large cage-like particles made from two separate saponin fractions, i.e., Matrix-A™ and Matrix-C™ (85% and 15%, respectively). The Matrix-A™ and -C™ particles are formed by formulating purified saponin fractions, Fr-A and Fr-C, respectively, from the tree Q. saponaria Molina with cholesterol and phospholipids.

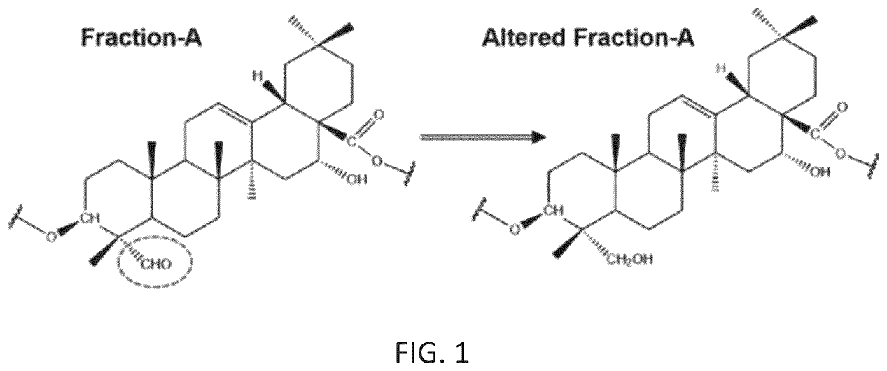

To track the biodistribution of saponins, all saponins in Fr-A were radiolabeled with tritium (3H). The aldehyde group on the quillaic acid triterpene aglycone (position C-23) was reduced to a hydroxyl group by tritium (3H)-labeled sodium borohydride [3H]-NaBH4 to obtain altered Fr-A material (FIG. 1A). For synthesis of deuterium (2H)-labeled Fr-A utilized in the adjuvanticity evaluation of the modified material, sodium borodeuteride (NaBD4) was used as reducing agent. The altered Fr-A was mixed with unmodified Fr-A at 50:50 ratio and then formulated into Matrix-A™ adjuvant particles. This was subsequently mixed with unmodified Matrix-C™ at an 85:15 ratio to form Matrix-M™ adjuvant [Matrix-M (3H-Sap)], also referred to as Matrix-M™ 50/50. To track the biodistribution of cholesterol, tritium labeled cholesterol [1-2-3H (N)] (Perkin Elmer, Bridgeport CT) was used to formulate radiolabeled Matrix-A™ particles, which were used together with unmodified Matrix-C™ to form Matrix-M™ adjuvant [Matrix-M (3H-Chol)].

SARS-COV-2 rS (construct BV2373, Novavax, Inc., Gaithersburg, MD) was constructed from the full-length, wildtype SARS-COV-2 S glycoprotein based upon the GenBank gene sequence MN908947 (nucleotides 21563-25384). The native full length S protein was modified by mutating RRAR to QQAQ (3Q) in the putative furin cleavage site located within the S1/S2 cleavage domain to become protease resistant. Two additional proline amino acid substitutions were inserted at positions K986P and V987P (2P) within the heptad repeat 1 (HR1) domain to stabilize SARS-COV-2 rS in a prefusion conformation. The synthetic transgene has been engineered into the baculovirus vector (BV2373) for expression in Spodoptera frugiperda (Sf9) insect cells to produce SARS-COV-2 rS proteins.

BALB/c mice were immunized subcutaneously at the base of the tail, at a total volume of 100 μL/mouse, on days 0 and 21. Three animal groups (each n=10) received 1 μg SARS-COV-2 rS either unadjuvanted or in combination with 5 μg Matrix-M™ adjuvant or 5 μg Matrix-M™ adjuvant containing modified (2H-labeled) Matrix-A™ (Matrix-M™ 50/50). Blood was collected from the lateral tail vein of fully conscious animals on day 20 and on day 28. Serum was extracted from the blood and stored at −20° C. until analysis.

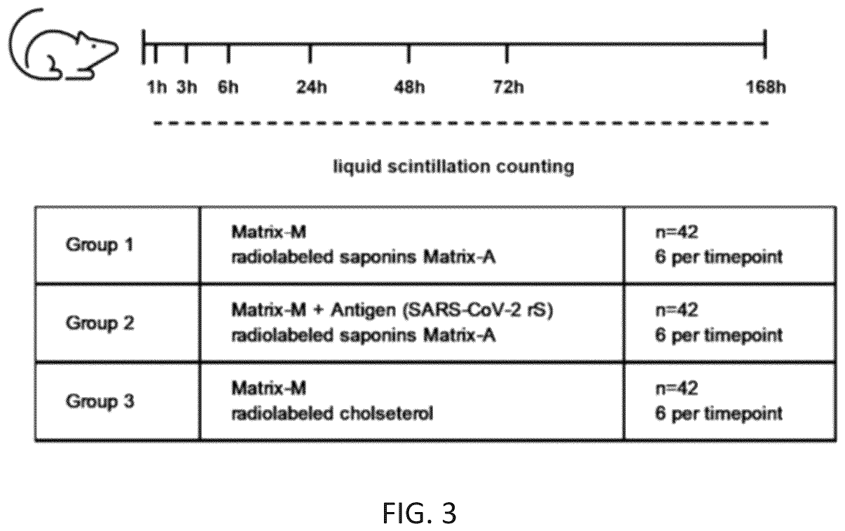

CD-1 IGS mice were immunized intramuscularly with a total volume of 40 μL into the right quadriceps femoris while anesthetized with isoflurane. Study groups and time points for termination are outlined in FIG. 3. Briefly, mice received a single dose of Matrix-M adjuvant (10 μg) containing radiolabeled saponins [Matrix-M (3H-Sap)], or Matrix-M adjuvant (10 μg) containing radiolabeled saponins and mixed with SARS-COV-2 rS (1 μg) [Matrix-M (3H-Sap)+SARS-COV-2 rS)], or Matrix-M adjuvant (10 μg) containing radiolabeled cholesterol and administered alone [Matrix-M (3H-Chol)] [each condition n=42, 6 mice (3 females, 3 males) per time point]. The weight of the syringe was recorded before and after the dosing to measure the weight of the injected solution. The radioactivity of the dosing formulation (DPM/g) was determined by liquid scintillation counting (MicroBeta2, Perkin Elmer), and the injected (radioactive) dose (ID) was calculated and used to determine the normalized radioactivity as % of injected dose per gram tissue (as described below).

On the day of sacrifice, animals were weighed and then euthanized. Samples were then collected in tared tubes and weighed. First, blood samples were collected using cardiac puncture to prepare plasma for activity analysis. Immediately after blood sampling, the animals were perfused with heparinized saline (2.5 IU/mL) and tissue samples weighing a maximum of 200 mg were collected. The following samples were collected: injection site (quadriceps femoris muscle, QF), iliac lymph nodes (LN), inguinal LN, popliteal LN, plasma, urine, kidney, liver, intestines, spleen, lungs, bone marrow, heart, brain, axillary LN, mandibular LN, mesenteric LN, testes, ovaries, and uterus. Right and left LN were pooled.

For the scintillation analysis of total radioactivity, the tissue samples were mixed with 0.1 mL 0.9% saline and then homogenized (TissueLyser II, Qiagen) with 30 s-1 frequencies for 2 min at +4° C. One mL Solvable (Perkin Elmer) was added to the homogenized tissues and samples were incubated for 2 h at +60° C. Finally, the solubilized tissue homogenate was diluted at a ratio of 1:10 in UltimaGold (Perkin Elmer). Samples of lymph nodes were not homogenized but directly dissolved in 0.5 mL Solvable. The processed tissue samples were analyzed in a microplate counter (MicroBeta2; Perkin Elmer).

Radioactivity of the samples was measured as disintegrations per minute (DPM) and converted to % of injected radioactivity/g of tissue (normalized % ID/g; relative radioactivity/activity) by the following formula: ((MD/(IS×DR))×100)/TW, where MD is the measured dose in DPM, IS is the amount of injected solution in grams (g), DR is the dose radioactivity in DPM/g and TW is tissue weight (g). The estimated limit of quantification (LOQ) was 703 DPM, which corresponds to a range of 0.0358-7.15% ID/g tissue after normalization, depending on the mass of the tissue sample analyzed. Samples with DPM values <LOQ were set to 703 DPM before the % ID/g was calculated.

Quantification of anti-S IgG1 and IgG2a antibodies in serum from day 20 and day 28 was performed by enzyme-linked immunosorbent assay (ELISA). Ninety-six-well MaxiSorp microplates (Nunc) were coated with 1.5 μg/mL SARS-COV-2 rS protein (BV2373) in PBS overnight at 4° C. Individual sera were serially diluted in PBS containing 0.05% Tween-20 (PBS-T) and 1% bovine serum albumin (BSA) in deep-well plates. Serum samples were serially diluted 5-fold in 8 steps, starting at 1:300 or 1:1000 (day 20), 1:300 or 1:10,000 (day 28) for IgG1, and 1:15, 1:100 or 1:500 (day 20), 1:15, 1:100 or 1:1000 (day 28) for IgG2a. The samples were then added to the antigen-coated microtiter plates in singlicate (day 20) or duplicate (day 28) and incubated for 2 hours at room temperature. Pooled sera from untreated BALB/c mice and sera from SARSCOV-2 rS (with Matrix-M adjuvant) immunized mice were used as negative and positive controls, respectively. After washing with PBS-T, diluted HRP-linked secondary antibody, anti-IgG1 or -IgG2a (BIORAD Laboratories, Hercules, CA, United States), was added and incubated for 2 hours at room temperature. After washing, TMB substrate was added and incubated for 10 min, and then the reaction was stopped using 1.8 M sulfuric acid. The absorbance was measured at 450 nm (SpectraMax M3, Molecular Devices). The anti-S titers were calculated using a four-parameter logistic equation (SoftMax software v.6.5.1). The inflection point of the titration curve (EC50 value) was defined using a 4-parameter logistic equation, based on the estimated curve fit. This was taken as the titer value. For a titer below the assay lower limit of detection (LOD), a titer of <15 (starting dilution) was reported and a value of “15” assigned to the sample to calculate the group mean titer.

hACE2 receptor blocking antibody titers were determined by ELISA. Ninety-six-well plates were coated with 1.0 μg/mL SARSCOV-2 rS protein overnight at 4° C. The coated wells were next blocked with StartingBlock™ (TBS) Blocking Buffer (ThermoFisher Scientific) for 1 h at room temperature. Mouse sera ere serially diluted 2-fold starting with a 1:20 dilution and were added to coated wells and incubated for 1 h at room temperature. After washing, 30 ng/ml of histidine-tagged hACE2 (Sino Biologics, Beijing, CN) was added to wells and incubated for 1 h at room temperature. HRP-conjugated anti-histidine IgG was added and incubated for 1 h, followed by addition of TMB substrate. The absorbance was measured at 450 nm with a SpectraMax Plus plate reader (Molecular Devices, Sunnyvale, CA, United States) and data were analyzed with SoftMax Pro 6.5.1 GxP software. Serum antibody titer at 50% inhibition (IC50) of hACE2 to SARS-COV-2 rS protein was then determined. For a titer below the assay lower limit of detection (LOD), a titer of half the value of the starting dilution (value of “10”) was assigned to the sample to calculate the group mean titer.

Data were analyzed statistically using GraphPad Prism software (version 9). For IgG1, IgG2a and hACE2 data, the geometric mean titer (GMT) with associated 95% confidence intervals (CI) were calculated by group, and the mean values of the log 10-transformed titer measurements were compared between groups using a one-way ANOVA with Tukey's multiple comparisons test. Comparisons of % ID/g or % ID/mL between the groups at different timepoints were analyzed by two-way ANOVA followed by Tukey's test. A p-value of <0.05 was considered statistically significant.

Flow cytometry experiment was carried out in BALB/c mice, which were immunized intramuscularly with a total volume of 50 μL into the right quadriceps femoris while anesthetized with isoflurane. The mice received a single dose of Matrix-M adjuvant (5 μg) in which 10% of Fraction-A in Matrix-A was fluorescently tagged. Time points for termination were 0.5 h, 1 h, 3 h, 6 h, 18 h, and 24 h. On the time point of sacrifice, 5 mice per time point were euthanized and the right quadriceps femoris muscle (injection site) and the right iliac lymph node (primary draining lymph node) collected. The samples were prepared into single cell suspensions and the cells labeled with fluorescent labelled antibodies as described in Table 1 and Table 2 and analyzed by flow cytometry for several different immune cell populations (neutrophils, monocytes/macrophages, dendritic cells, and lymphocytes). Flow cytometry analysis was done with FlowJo v10 10.9.0 (BD Life Sciences).

TABLE 1

| Population | Definition | Full gating path |
|---|---|---|
| Immune cells | CD45⁺ | cells/Single Cells/Single Cells/live |
| | | cells/CD45+ |
| Neutrophils | CD45⁺CD11b^{hi}Ly6G^{hi} | cells/Single Cells/Single Cells/live |
| | | cells/CD45+/neutrophils |
| T cells | CD45⁺CD11b^{+/-}Ly6G^{lo/-}CD3⁺ CD49b⁻ | cells/Single Cells/Single Cells/live |
| | | cells/CD45+/neutrophils−/CD3+/T cells |
| NKT cells | CD45⁺CD11b^{+/-}Ly6G^{lo/-}CD3⁺ CD49b⁺ | cells/Single Cells/Single Cells/live |
| | | cells/CD45+/neutrophils−/CD3+/NKT cells |
| Monocytes | CD45⁺CD11b^{+/-}Ly6G^{lo/-}CD3⁻Ly6c⁺F4/80⁻ | cells/Single Cells/Single Cells/live |
| | | cells/CD45+/neutrophils−/CD3−/Q1: F4_80−, |
| | | Ly6C+ |
| F4/80+ | CD45⁺CD11b^{+/-}Ly6Glo/⁻ | cells/Single Cells/Single Cells/live |
| monocytes | CD3⁻Ly6c⁺F4/80⁺ | cells/CD45+/neutrophils−/CD3−/Q2: F4_80+, |
| | | Ly6C+ |
| DCs: All | CD45⁺CD11b^{+/-}Ly6G^{lo/-} | cells/Single Cells/Single Cells/live |
| | CD3⁻Ly6c⁻F4/80⁻ | cells/CD45+/neutrophils−/CD3−/Q4: F4_80−, |
| | CD11c⁺MHCIIhigh | Ly6C−/DCs |
| DCs: | CD45⁺CD11b^{+/-}Ly6G^{lo/-} | cells/Single Cells/Single Cells/live |
| CD11b+ | CD3⁻Ly6c⁻F4/80⁻ | cells/CD45+/neutrophils−/CD3−/Q4: F4_80−, |
| | CD11c⁺MHCII^{high}CD11b⁺ | Ly6C−/DCs/CD11b+ |
| DCs: CD11b− | CD45⁺CD11b^{+/-}Ly6G^{lo/-} | cells/Single Cells/Single Cells/live |
| | CD3⁻Ly6c⁻F4/80⁻ | cells/CD45+/neutrophils−/CD3−/Q4: F4_80−, |
| | CD11c⁺MHCII^{high}CD11b⁻ | Ly6C−/DCs/CD11b− |
| B cells* | CD45⁺CD11b^{+/-}Ly6G^{lo/-} | cells/Single Cells/Single Cells/live |
| | CD3⁻Ly6c⁻F4/80⁻CD11c⁻ | cells/CD45+/neutrophils−/CD3−/Q4: F4_80−, |
| | MHCII⁺CD3⁻ | Ly6C−/DCs−/B cells |
| NK cells | CD45⁺CD11b^{+/-}Ly6G^{lo/-} | cells/Single Cells/Single Cells/live |
| | CD3⁻Ly6c⁻F4/80⁻CD11c⁻ | cells/CD45+/neutrophils−/CD3−/Q4: F4_80−, |
| | MHCII⁻CD3⁻CD49b⁺ | Ly6C−/DCs−/MHCII−/NK cells |
| Others | CD45⁺CD11b^{+/-}Ly6G^{lo/-} | cells/Single Cells/Single Cells/live |
| | CD3⁻Ly6c⁻F4/80⁻CD11c⁻ | cells/CD45+/neutrophils−/Q4: F4_80−, Ly6C−/ |
| | MHCII⁻CD3⁻CD49b⁻ | DCs−/MHCII−/others |

*Note that there is no designated B cell marker included in the panel. B cells are instead defined as being MHCII+ cells that are neither monocytes, macrophages, nor DCs, and that also are negative for neutrophil as well as T− NKT−, and NK cell markers. These cells also show lymphocytic characteristics in a FSC vs SSC plot

TABLE 2
Antibody

| Target | Fluorophore | Clone |
|---|---|---|
| CD3 | BV421 | 145-2C11 |
| Ly6G | BV510 | 1A8 |
| F4/80 | BV605 | T45-2342 |
| CD11b | BV650 | M1/70 |
| I-A/I-E (MHCII) | BV786 | M5/114.15.3 |
| CD49b | PerCP-Cy5.5 | DX5 |
| CD11c | PE | HL3 |
| CD45 | PE-Cy7 | PE-Cy7 |
| Ly6C | R718 | AL-21 |

To evaluate the cytokine and chemokine response induced by an adjuvant together with the quadrivalent Nanoparticle Influenza Vaccine (NIV) (A/Michigan, A/Singapore, B/Phuket, B/Iowa), a total of 125 female BALB/c mice (n=25/group), 8 weeks old, were immunized intramuscularly in the hind leg with 6 μg hemagglutinin (HA)/strain NIV together with any of the three adjuvants; 5 μg Matrix-M1 adjuvant, 3 μg AS01B, or 200 μg Alum. Ag (NIV) alone and PBS were used as controls. Five mice per group were sacrificed 6, 24, 48, 72, and 168 hours post primary immunization (p.p.i.) to collect injection site (muscle), and draining lymph node (dLN) samples.

The tissues were collected in tubes and snap-frozen in a dry shipping container chilled and saturated with liquid nitrogen until the tissues were homogenized to extract soluble proteins. Briefly, the tissues were placed in cold PBS solution containing protease inhibitors (Halt™ Protease inhibitor cocktail, Thermo Scientific, Gothenburg, Sweden) and homogenized on ice with a tissue homogenizer as quickly as possible to avoid heating the sample. To collect supernatants, the samples were briefly spun down and then transferred to microtubes for further centrifugation at 10,000×g for 10 minutes at 4° C. The dLN samples were centrifuged once and the muscle samples twice, transferring the supernatants to new tubes after each centrifugation. The supernatants were then stored at −70° C. until further analysis.

Cytokine and chemokine concentrations in the samples were analyzed with the V-PLEX Plus Mouse Cytokine 19-Plex Kit from Meso Scale Discovery (MSD; Rockville, MD, USA) by electrochemoluminescence at the Clinical Biomarkers Facility at SciLifeLab, Uppsala University, Sweden. Prior to analysis, the samples were diluted according to the manufacturer's instructions. The following analytes were measured on a MESO QuickPlex SQ 120: IFN-Y, IL-1B, IL-2, IL-4, IL-5, IL-6, IL-9, IL-10, IL-12p70, IL-15, IL-17A/F, IL-27p28/IL-30, IL-33, IP-10, KC/GRO, MCP-1, MIP-1a, MIP-2, and TNF-α.

### Results

Experiment 1: Adjuvanticity of Matrix-M™ Containing Matrix-A™ with Partially Modified Saponins

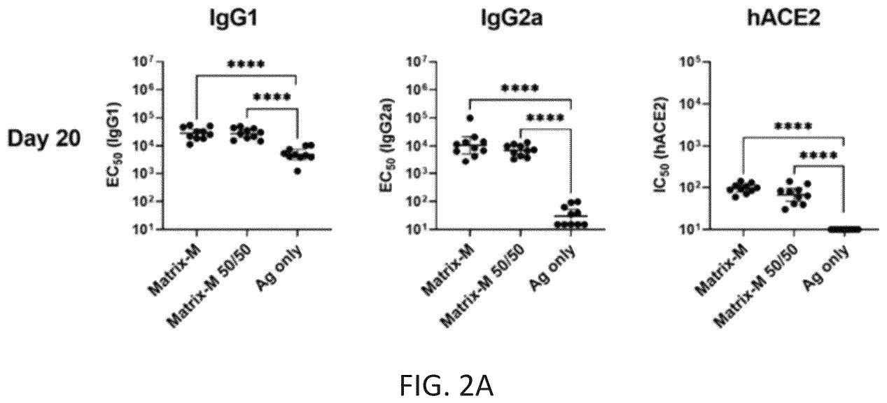

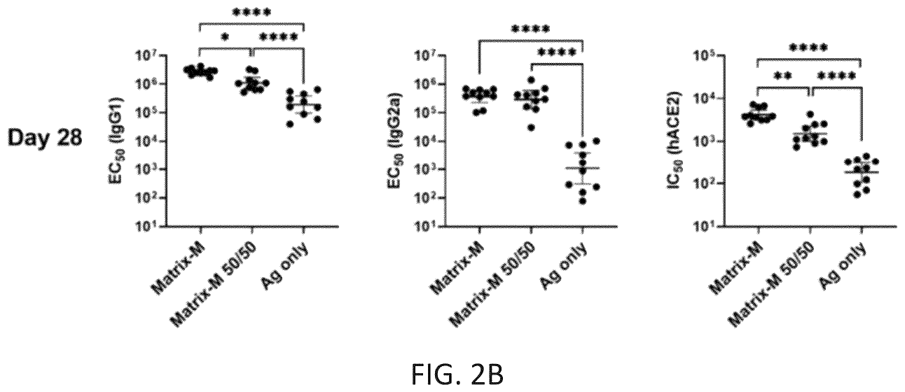

Tracking the biodistribution of saponins after immunization with Matrix-M™ adjuvant was enabled by radiolabeling Fr-A by reduction of the aldehyde substituent on the quillaic acid triterpene aglycone (position C-23), FIG. 1. The adjuvanticity of unmodified Matrix-M™ adjuvant in comparison to Matrix-M™ formulated from 50% altered and 50% unaltered Fr-A material (Matrix-M™ 50/50) was evaluated in a 2-dose immunization scheme with SARS-COV-2 rS antigen in BALB/c mice to confirm retention of adjuvanticity using the scheme illustrated in FIG. 2. At day 20 post primary immunization, similar titers of SARS-COV-2 rS-specific IgG1 and IgG2a as well as similar titers of functional human angiotensin converting enzyme two (hACE2) receptor blocking antibodies were found in sera of both adjuvanted groups, FIG. 3A, with both clearly superior to unadjuvanted antigen. At day 28, the IgG1 anti rS, along with the hACE2 receptor inhibiting antibody response, were somewhat reduced in the group that had received SARS-COV-2 rS adjuvanted with Matrix-M™ 50/50, FIG. 3B, while IgG2a anti-rS titers remained essentially the same in both adjuvanted groups. The comparison to unadjuvanted SARS-COV-2 rS immunization showed an adjuvant effect of both unmodified Matrix-M™ and Matrix-M™ 50/50 regarding rS-specific IgG1 and IgG2a as well as hACE receptor blocking antibodies at day 20 and day 28. Thus, the clear adjuvant function of Matrix-M™ 50/50 as assessed by humoral immunity, albeit marginally reduced, suggested the suitability of modified Matrix-M™ 50/50 to represent Matrix-M™ adjuvant to follow the biodistribution of saponins. Of interest, the Matrix-M™ containing labeled saponins showed no reduction in its capacity to induce antigen-specific IgG2a responses, a marker of the known tendency of Matrix-M™ to support strongly Th1-biased T cell responses.

### Experiment 2: Local Biodistribution

To study the local biodistribution and particle integrity of Matrix-M™ adjuvant after intramuscular injection in CD-1 mice, radioactivity in the quadriceps femoris muscle (QF; injection site) and lymph nodes (LN) draining the hind limb was analyzed at seven timepoints starting at 1 h and continuing to 168 h (7 days) after injection of Matrix-M™ adjuvant containing either radiolabeled saponins (+/−without SARSCOV-2 rS antigen) or cholesterol.

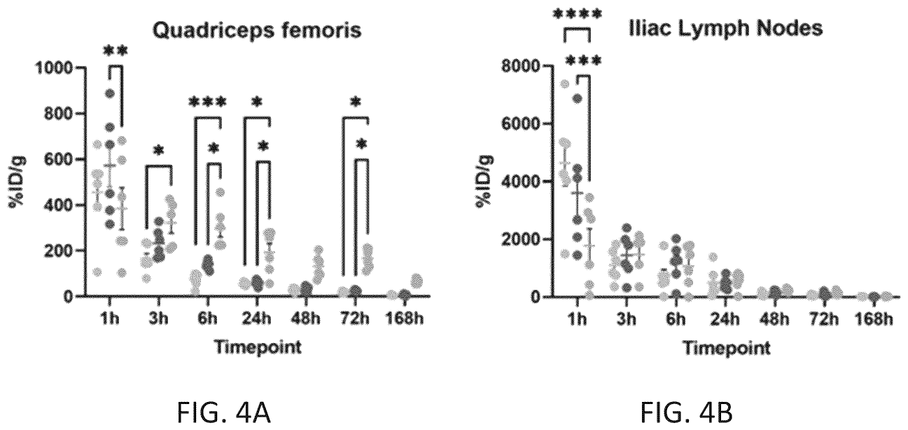

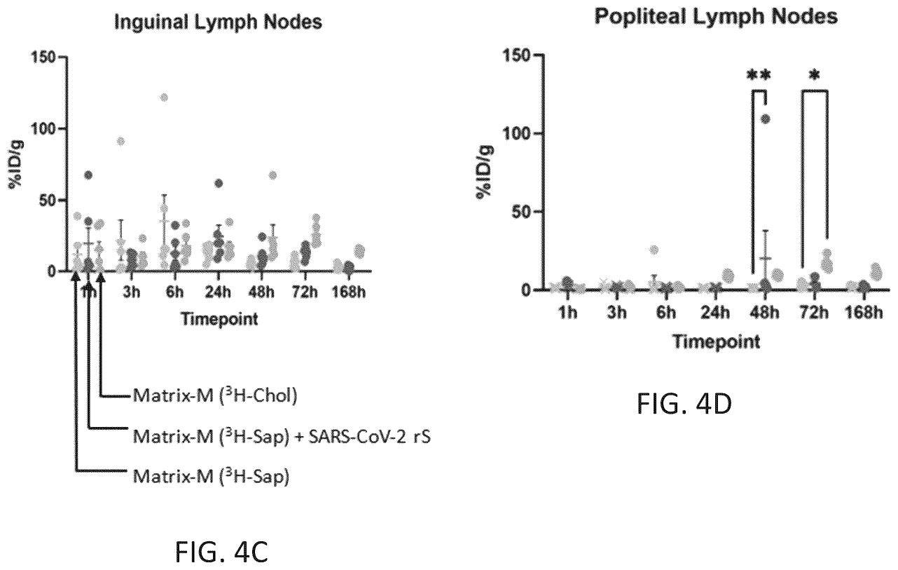

One hour post injection (p.i.), a high percentage of injected radioactive dose per gram tissue (% ID/g) for both saponins and cholesterol was detected at the injection site and in the iliac LN. The distribution at the QF injection site, iliac lymph nodes, inguinal lymph nodes and popliteal lymph nodes is shown in FIGS. 4A, 4B, 4C, and 4D, respectively. Distribution to the iliac LN was substantial as compared to distribution to the inguinal LN and popliteal LN, thus indicating the iliac LN as the primary draining LN (dLN) after QF injection. Saponins in the QF and the iliac LN showed a fast decline starting from 1 h p.i. At 48 h p.i., saponins reached low levels in both the QF and the iliac LN and remained low for the remainder of the experiment. At the injection site, cholesterol counts showed a slower decline compared to saponin counts, leading to significantly higher cholesterol than saponin levels starting at 3-6 h p.i., suggesting that Matrix particles had been at least partially disassembled at these timepoints. The presence or absence of SARS-COV-2 rS antigen was found to have no effect on the local biodistribution of saponins.

### Experiment 3: Systemic Biodistribution

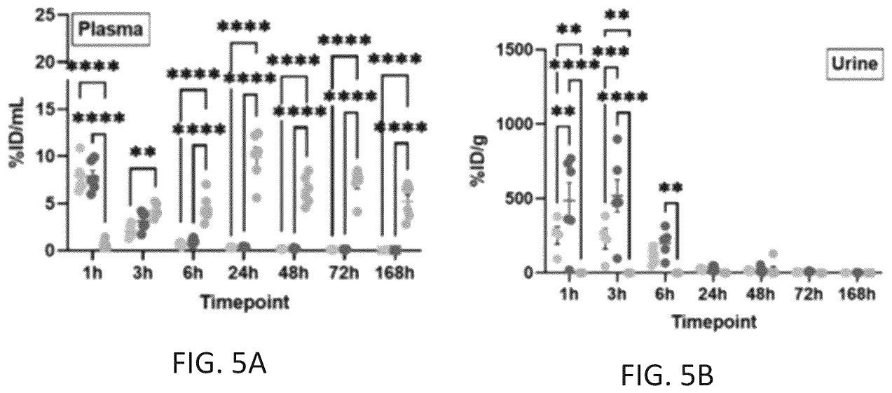

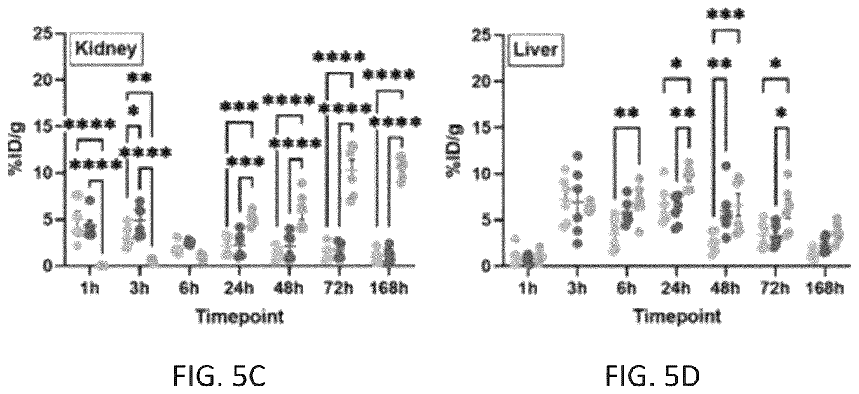

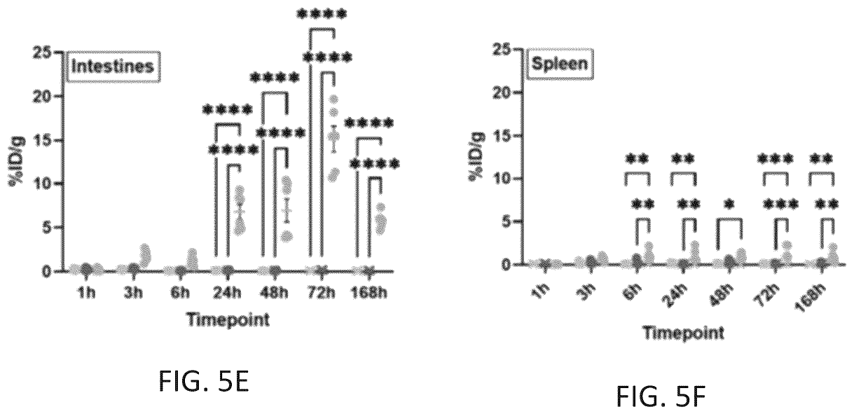

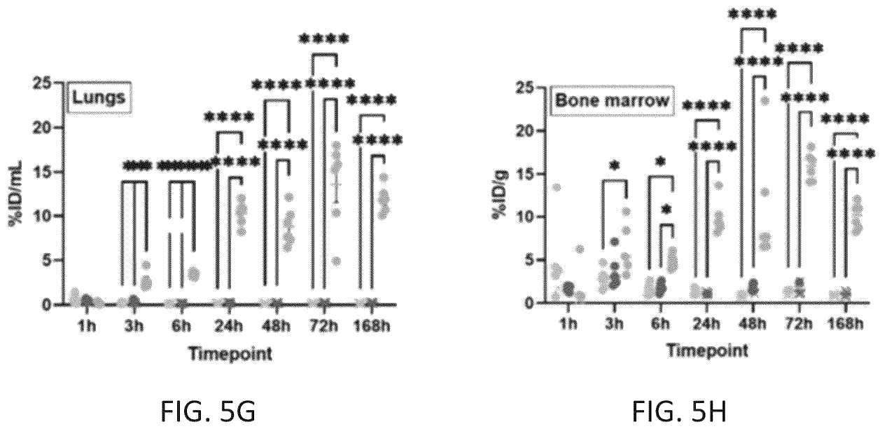

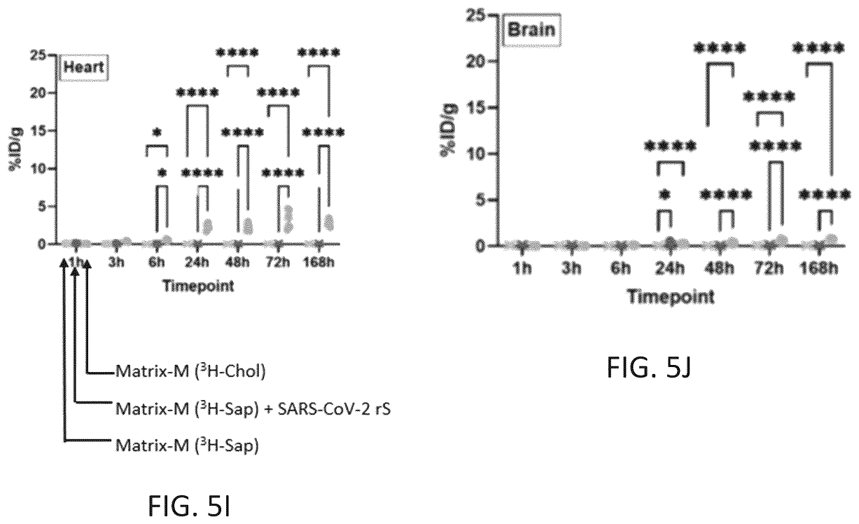

Body fluids and non-local tissues were studied to understand both the systemic distribution of Matrix-M™ adjuvant and its constituent compounds, and to indicate possible excretion pathways and their time course. FIGS. 5A-J show the distribution to different sites within the body over time. The retrieved relative radioactivity of labeled saponins differed from that of cholesterol at most timepoints in plasma, urine, and kidney, FIGS. 5A-C, respectively. The relative activity of labeled saponins in plasma, urine, and kidney peaked at 1-3 h p.i. followed by a rapid decline at 6 h p.i. In contrast, lower relative radioactivity of cholesterol was detected early in plasma and kidney followed by an increase to a plateau at around 5%-20% ID/g at 24 h p.i. which persisted until the last measurement at 168 h p.i., comparable to findings in the non-draining LN. Barely detectable activity of cholesterol was observed in urine, which is as expected for a lipophilic compound such as cholesterol. Of note, the presence of SARS-COV-2 antigen in the immunization led to an increased relative activity of saponins in urine at 1 h and 3 h p.i. and in kidney at 3 h p.i. In contrast, the absence or presence of antigen had no effect on the saponin activity in plasma, liver, intestines, spleen, lungs, bone marrow, heart, and brain at any study timepoint.

A unique pattern of relative activity of saponins in relation to cholesterol was found in the liver, FIG. 5D, with low levels at 1 h p.i. for both labeled compounds, which then increased to a similar activity for both at 3 h p.i. followed by a slightly higher relative activity of cholesterol between 6 and 72 h p.i . . .

The bone marrow exhibited a peak of saponins at 3 h and 6 h p.i., which declined at 24 h p.i., FIG. 5H. Low relative activity of saponins, partially below LOQ, were found in intestines, spleen, lungs, heart, and brain. Intestines, lungs, and bone marrow, FIGS. 5E, 5G and 5H, showed an increase in the relative activity of cholesterol, reaching a plateau of around 5-20% ID/g tissue at 24 h p.i., which was stable through 168 h p.i., with comparable levels found in non-draining LN, liver, and kidney. A similar pattern with plateauing cholesterol activity from 24 h p.i. was found in spleen, heart, and brain, albeit at lower levels, FIGS. 5F, 5I and 5J.

Thus, these analyses suggest a rapid and substantial disassembly of Matrix particles in vivo as demonstrated by a different biodistribution of saponins and cholesterol. Saponins in Matrix-M™ adjuvant do not accumulate systemically but are rapidly excreted via the kidneys into urine. In contrast, systemic cholesterol levels reached a plateau at around 24 h p.i. that remained stable until the end of measurement (168 h p.i.). This suggests that, as the particle disassembles, labeled cholesterol enters the body's cholesterol recycling pool before gradual excretion/metabolism.

### Experiment 4: Biodistribution to the Reproductive Tract

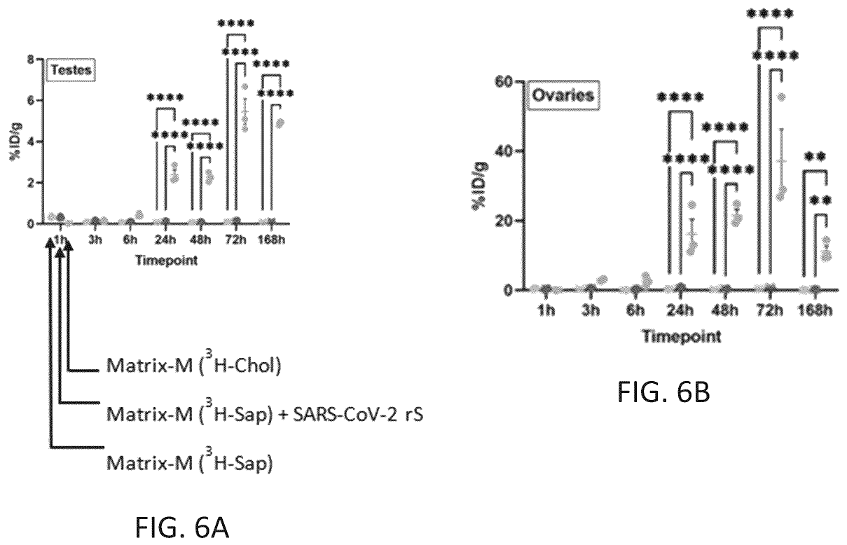

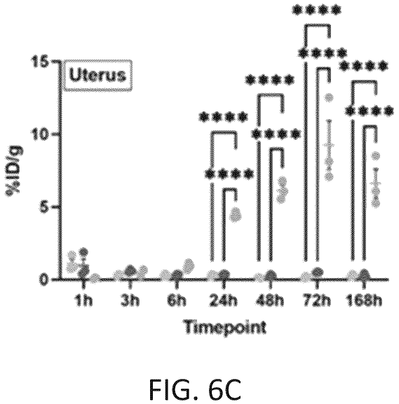

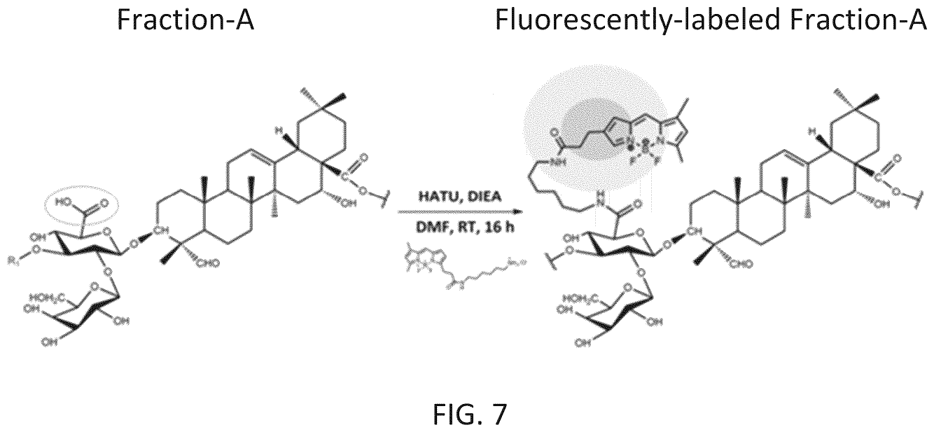

To evaluate the biodistribution of labeled saponins and labeled cholesterol in organs of the reproductive tract after Matrix-M™ adjuvant injection, testes from male mice as well as ovaries and uterus from female mice were analyzed. FIGS. 6A-6C respectively show the distribution of labeled saponins and labeled cholesterol in the testes, ovaries and uterus. The relative activity of saponins was low in all three organs with the highest relative activity detected at 1 h p.i., which then declined at 3 h p.i. to remain low until the last measurement at 168 h p.i . . . . Adding the SARS-COV-2 rS antigen to the adjuvant had no effect on the relative saponin activity in the reproductive organs of either males or females. The pattern of the relative cholesterol activity in all three organs reached a plateau starting from 24 h p.i. with the highest relative activity of cholesterol detected in the ovaries. Overall, the saponin and cholesterol distribution patterns to the reproductive tract were comparable to those found in the other analyzed solid organs.

### Experiment 5: Cellular Distribution of Matrix-MIM Adjuvant

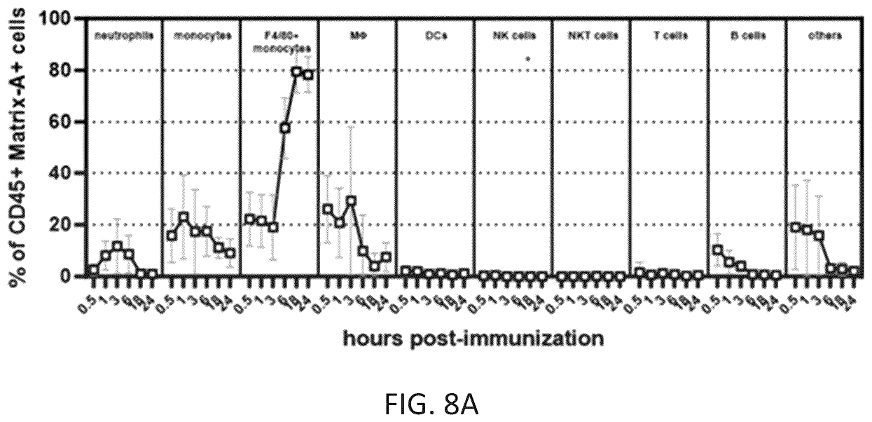

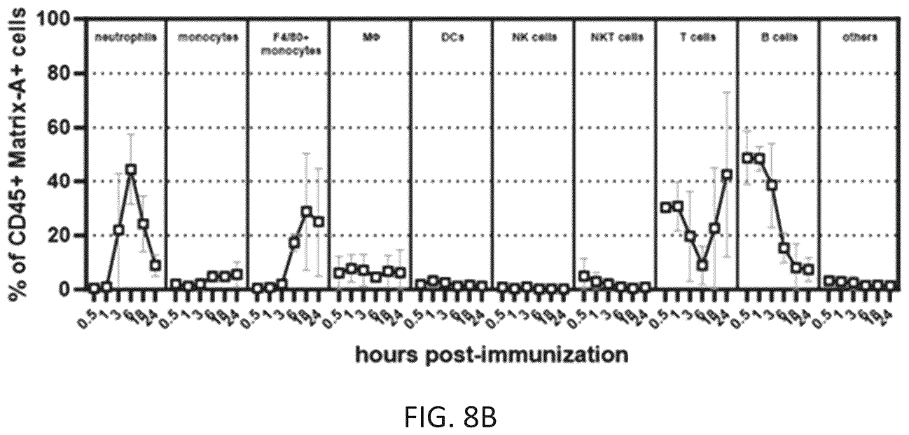

In an experiment to determine which cells take up the Matrix-M™ adjuvant, mice were injected with antigen and fluorescently-modified Matrix-M™ adjuvant. The injection site muscular tissue and the dLN were harvested at 0.5 h, 1 h, 3 h, 6 h, 18 h, and 24 h p.i., and various cells from the tissue samples were counted by flow cytometry. FIG. 8A shows graphs of percentage of a type of cell taken from the injection site that were both CD45 positive and positive for the fluorescently tagged Matrix-M™. Results are shown for ten different types of cell, namely cells, natural killer T (NKT) cells, B cells and others. The graphs show, for example, that about 80% of the F4/80-monocytes were positive for CD45 and fluorescently tagged Matrix-M™ at a time 18 h p.i. While neutrophils, monocytes, in particular F4/80-monocytes, and macrophages at the injection site showed Matrix-M™ association, there was negligible uptake in DCs, NK cells, NKT cells, T cells and only minimal association with B cells.

FIG. 8B shows the same data for cells derived from the dLN. In contrast to injection site cells, there was a higher percentage of Matrix-M™ tagged neutrophils and a much lower percentage of Matrix-M™ tagged monocytes. There was also significant association with T cells and B cells, which did not occur at the injection site.

### Experiment 6: Cytokine Induction at Injection Site and dLN

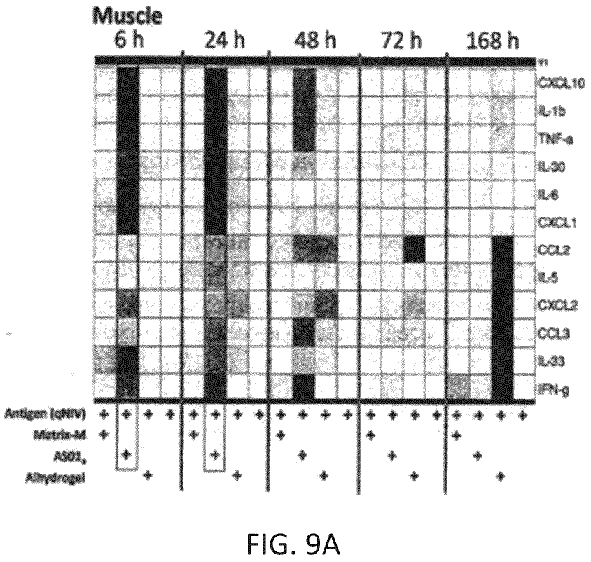

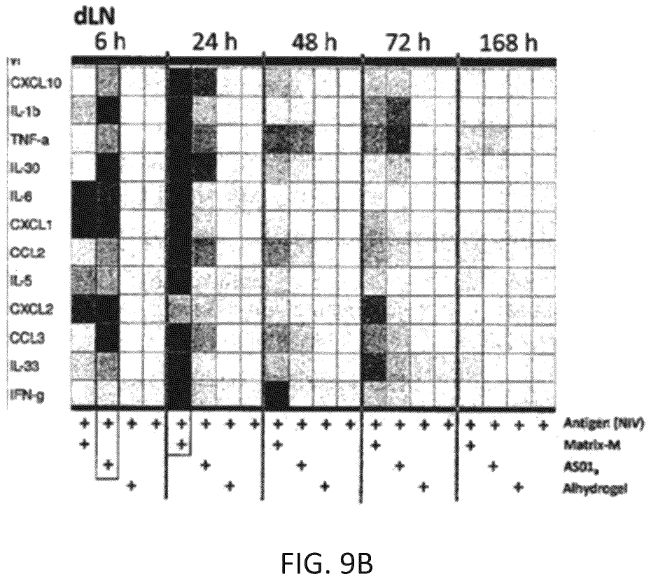

FIG. 9A shows a heat map of cytokines induced as a function of time at the muscle injection site for various antigen/adjuvant combinations. The antigen, (qNIV) was injected with Matrix-M™, AS01, or alhydrogel, or was injected without any adjuvant. The data are shown for five different times: 6 h, 24 h, 48 h, 72 h, and 168 h. Each time on the heat map includes four columns, each column relating to the adjuvant used. The cytokines examined were CXCL10, IL-1b, TNF-α, IL-30, IL-6, CXCL1, CCL2, IL-5, CXCL2, CCL3, IL-33, and IFN-g. In the muscle, it was found that stronger cytokine responses were obtained with the AS01 composition at 6 h and 24 h. However, at 168 h, the alhydrogel composition induced the greater response, at least for some cytokines.

FIG. 9B shows a similar heat map, but for cytokines induced in the dLN. Like the muscle, the AS01 composition induced the greatest cytokine response at 6 h. The Matrix-M™ composition showed a reduced response at 6 h, while the alhydrogel composition and the composition containing antigen alone showed negligible cytokine response. At 24 h, however, the Matrix-M™ composition induced a significant response across the spectrum of cytokines, a broader response than the AS01 composition was able to produce at any time.

### DISCUSSION

Monitoring the biodistribution of Matrix-M™ adjuvant, a key component of NVX-CoV2373 and several novel vaccine candidates, is an important step in elucidating the Matrix-M™ adjuvant method of action (MoA) and safety profile. The use of Matrix-M™ adjuvant composed of unlabeled Matrix-C™ particles and Matrix-A™ particles containing either radiolabeled saponins or radiolabeled cholesterol, enabled the demonstration of a very rapid transfer of Matrix-M™ adjuvant from the injection site and the dLN after intramuscular injection. Typically, the dLN demonstrated a peak in the labeled components within one hour p.i. A sharp reduction of both labeled components was found at the injection site and in the dLN within 48 h, together with a transient increase of saponin counts, but not of cholesterol, in plasma, urine, and kidneys at early timepoints. Low levels of relative cholesterol radioactivity were detected systemically between 24 h and the latest measurement of the study (168 h). In general, the biodistribution of saponins was independent of the presence of the rS nanoparticle antigen in the injection. This demonstrates the biodistribution of saponins and cholesterol of Matrix-ATM, the main component of Matrix-M™, after immunization with Matrix-M™. Due to the low content of Matrix-C™ in Matrix-M™, radiolabeling of Matrix-C components is not sufficient for detection. Therefore, a slightly different biodistribution profile for Matrix-C™, e.g., in its kinetics cannot be excluded.

The fast removal of saponins and cholesterol from the injection site within 48 h p.i. together with an early distribution of saponins and cholesterol to the dLN suggests that the MoA of Matrix-M™ adjuvant is independent of a depot effect. Instead, the rapid distribution of saponins and cholesterol to the iliac LN further strengthens the concept of Matrix-M™ adjuvant as “setting the stage” for the antigen in the dLN. These results are in line with a comparison of adjuvant-active and attenuated minimal QS-21 saponin adjuvants demonstrating a preferential localization of the active QS-21 in the draining lymph node at 24 h p.i. Of note, in the presented radioactivity data, the iliac LN (and all other LN) from the injected and opposing site were pooled together, most likely diluting the observed effect as only changes at the draining site can be expected.

The present finding of the rapid transfer of Matrix-M™ adjuvant to the dLN is also consistent with previously published data describing Matrix-M™ as a potent inducer of immune cell migration (mainly monocytes, DCs, and neutrophils) to the dLN shortly after injection of Matrix-M™ adjuvant with or without antigen. These cells had acquired an activated phenotype as required to initiate antigen specific adaptive immunity; an effect that was more pronounced for Matrix-M™ adjuvant in a head-to-head comparison with Alhydrogel, Freund's Complete Adjuvant and AS03 at 48 h post subcutaneous immunization.

The exact mechanisms by which Matrix-M™ adjuvant and antigen reach the dLN, e.g., the relative contributions of cellular transport and free flow through the lymph, remain to be investigated in future studies.

Although the data presented here show a fast reduction of either of the two labeled Matrix components, saponin and cholesterol, within 48 h in the QF, the more rapid removal of saponins than cholesterol from QF suggests a significant disassembly of the Matrix particles already at the injection site. The strong affinity between saponins and cholesterol is well recognized and cholesterol is crucial for the formation of Matrix particles, which are stable on storage in neutral buffers. However, the fate of the Matrix particles in vivo is probably different. It is believed that, similar to other saponin-based adjuvants such as ISCOMATRIX and AS01, the Matrix particles target phagocytic cells and are processed by the endosomal/lysosomal pathway, and that such processing is dependent on both the acidification of and the enzymatic activities present in the lysosome. In this intracellular processing, the Matrix particles are likely disassembled at lower pH within the lysosome, liberating the saponins. As has been demonstrated for other saponin-based adjuvants, a key consequence of the liberation of the saponins is the destabilization of the lysosomal membrane. Lysosomal membrane permeabilization then provides access of the coadministered vaccine antigens to the cytosol, thus allowing cross-presentation by MHC class I molecules and the induction of antigenspecific CD8+ T cell responses. Such responses have been demonstrated for Matrix-M™ adjuvanted vaccines in murine models and in humans upon vaccination with NVX-CoV2373.

In addition, the transient peak detection of saponins but not of cholesterol in plasma, urine, and kidneys before 24 h p.i. confirms the distinct excretion pathways of non-associated cholesterol and saponin and their respective degradation products. In the liver, similar relative saponin and cholesterol activities were detected at 1 and 3 h p.i. However, this does not necessarily imply that Matrix particles are intact in this particular organ, as other studied fluids and tissues indicated at least a partial disassembly of Matrix particles at these timepoints. Saponins and possible degradation products appeared rather quickly in urine with a near complete clearance within 7 days, thus indicating this as a major excretion pathway. In contrast, cholesterol was detected systemically between 24 h and the latest measurement of the study (168 h), although the systemic cholesterol radioactivity was low in comparison to initial activities in the QF and the iliac LN. It can be speculated that at least a part of the labeled cholesterol enters the cholesterol recycling pool as an essential component of cell membranes, which may explain the systemic detection and slower clearance kinetics. Due to technical limitations, it was not possible to analyze feces in this study, which generally represents a major pathway for the elimination of cholesterol.

Generally, no differences in the saponin biodistribution dependent on the presence or absence of the rS nanoparticle antigen were found, as also described for other antigens and minimal QS-21. This would be expected because, in contrast to the saponin-based adjuvant ISCOM in which the antigen is physically incorporated into the particle, Matrix formulations consist of formulated particles that are mixed with antigen. However, at a few timepoints, the presence of rS antigen in the immunization led to an increased relative activity of saponins in the urine (1 h and 3 h p.i.) and in the kidney (3 h p.i.). As these differences are minor and rare the overall interpretation is that the presence or absence of vaccine antigen does not substantially influence the biodistribution of saponins.

The localized and time-restricted occurrence of Matrix-M™ adjuvant at the injection site and its rapid distribution to the dLN is not only favorable for the efficient induction of a memory response specific for the co-administered antigen but also from a safety profile perspective. The observed rapid clearance of saponins without a general systemic distribution can be incorporated into the benefit-risk assessments of Matrix-M™-adjuvanted vaccines. Of note, no long-term accumulation of saponins in any organ (including the reproductive tract) was observed. It is reasonable to believe that the lack of complete elimination of cholesterol after 168 h can be explained by cholesterol from the Matrix-particles entering the endogenous pool as essential components of cell membranes or metabolites. The described overall favorable safety profile from a biodistribution perspective confirms the results of a mild to moderate and transient reactogenicity of numerous phase 1-3 clinical trials evaluating vaccines containing Matrix-M™ adjuvant against COVID-19, malaria, influenza, or Ebola Virus Disease.

In summary, the data presented here demonstrate a rapid transfer of Matrix-M™ to the dLN, followed by a rapid removal of detected saponins and cholesterol from both the injection site and the dLN. Importantly, this transient presence of the adjuvant in the dLN is sufficient for the generation of high titers of neutralizing antibodies and immune memory, and an IgG2a signature consistent with a strong Th1 T cell response. The head-to-head comparison of saponins and cholesterol as critical components of Matrix particles indicate a fast particle disassembly at the injection site, which may be crucial for intracellular events like the lysosomal membrane permeabilization required for the reported CD8+ T cell activation in response to Matrix-M™-adjuvanted antigens. Additionally, the results show the important differences in the immune response at the dLN compared to the injection site.

Various modifications, equivalent processes, as well as numerous formulations to which the present invention may be applicable will be readily apparent to those of skill in the art to which the present invention is directed upon review of the present specification. The claims are intended to cover such modifications and processes.

As noted above, the present invention is applicable to saponin-based adjuvants. Accordingly, the present invention should not be considered limited to the particular examples described above, but rather should be understood to cover all aspects of the invention as fairly set out in the attached claims.

### Embodiments of the Disclosure

Specific enumerated embodiments <1> to <30>provided below are for illustration purposes only, and do not otherwise limit the scope of the disclosed subject matter, as defined by the claims. These enumerated embodiments encompass all combinations, sub-combinations, and multiply referenced (e.g., multiply dependent) combinations described therein.

1. A method of providing a vaccination composition to a mammalian subject, comprising:
injecting the vaccination composition at an intramuscular injection site of the subject, the vaccination composition comprising a saponin-based adjuvant and the intramuscular injection site being associated with a draining lymph node (dLN);
wherein, following injection of the vaccination composition at the intramuscular injection site, a fraction of T cells in the dLN that are positive for i) CD45 and ii) the saponin-based adjuvant has a minimum value at a time greater than one hour following injection of the vaccination composition at the intramuscular injection site and less than 24 hours following injection of the vaccination composition at the intramuscular injection site, the fraction of T cells in the dLN that are positive for i) CD45 and ii) the saponin-based adjuvant at a time 24 hours following injection of the vaccination composition having a value greater than the minimum value.

2. The method of embodiment 1, wherein the minimum value is more than about one tenth of a maximum value of the fraction of T cells in the dLN that are positive for i) CD45 and ii) the saponin-based adjuvant between one hour and twenty four hours following injection of the vaccination composition at the intramuscular injection site.

3. The method of embodiment 1, wherein the minimum value is more than about one eighth of a maximum value of the fraction of T cells in the dLN that are positive for i) CD45 and ii) the saponin-based adjuvant between one hour and twenty four hours following injection of the vaccination composition at the intramuscular injection site.

4. The method of embodiment 1, wherein the minimum value is more than about one fifth of a maximum value of the fraction of T cells in the dLN that are positive for i) CD45 and ii) the saponin-based adjuvant between one hour and twenty four hours following injection of the vaccination composition at the intramuscular injection site.

5. The method of any one of embodiments 1-4, wherein the saponin-based adjuvant comprises nanoparticles of Q. saponaria saponin fraction A, cholesterol and phospholipid and nanoparticles of Q. saponaria saponin fraction C, cholesterol and phospholipid.

6. The method of any of embodiments 1-5, wherein the mammalian subject is a human.

7. The method of any of embodiments 1-6, wherein the saponin-based adjuvant is mixed with an antigen.

8. The method of any of embodiments 1-7, wherein the saponin-based adjuvant is mixed with a carrier.

9. A method of providing a vaccination composition to a mammalian subject, comprising:
injecting the vaccination composition at an intramuscular injection site of the subject, the intramuscular injection site being associated with a draining lymph node (dLN), the vaccination composition including a saponin-based adjuvant;
wherein, over a time period between about one hour and about twenty four hours following injection of the vaccination composition, a maximum value of a fraction of B cells in the dLN that are positive for i) CD45 and ii) the adjuvant occurs at a time less than three hours following injection of the mixture at the intramuscular injection site.

10. The method of embodiment 9, wherein a value of the fraction of B cells in the downstream lymph node positive for i) CD45 and ii) the adjuvant that are B cells at about twenty four hours following injection of the mixture at the intramuscular injection site is less than about one tenth of the maximum value.

11. The method of embodiment 9, wherein a value of the fraction of cells in the downstream lymph node positive for i) CD45 and ii) the adjuvant that are B cells at about twenty four hours following injection of the mixture at the intramuscular injection site is less than about one twentieth of the maximum value.

12. The method of any of embodiments 9-11, wherein the saponin-based adjuvant comprises nanoparticles of Q. saponaria saponin fraction A, cholesterol and phospholipid and nanoparticles of Q. saponaria saponin fraction C, cholesterol and phospholipid.

13. The method of any of embodiments 9-12, wherein the mammalian subject is a human.

14. The method of any of embodiments 9-13, wherein the saponin-based adjuvant is mixed with an antigen.

15. The method of any of embodiments 9-14, wherein the saponin-based adjuvant is mixed with a carrier.

16. A method for administering a vaccination composition to a mammalian subject, comprising:
injecting the vaccination composition at an intramuscular injection site of the subject, the intramuscular injection site being associated with a draining lymph node (dLN);
wherein the vaccination composition comprises an antigen and a saponin-based adjuvant, the saponin-based adjuvant comprising a saponin component and a cholesterol component; and
wherein, following injection of the vaccination composition at the intramuscular injection site of the subject, concentration of the saponin component in the subject's blood plasma peaks at a time less than 3 hours after the injection, and concentration of the cholesterol component in the subject's blood plasma peaks at a time greater than 6 hours after the injection.

17. The method of embodiment 16, wherein the saponin-based adjuvant comprises nanoparticles of Q. saponaria saponin fraction A, cholesterol and phospholipid and nanoparticles of Q. saponaria saponin fraction C, cholesterol and phospholipid.

18. The method of any of embodiments 16-17, wherein the saponin-based adjuvant is mixed in a carrier.

19. The method of claim any of embodiments 16-18, wherein, following injection of the vaccination composition at the intramuscular injection site of the subject, concentration of the saponin component in the subject's kidney peaks at a time less than 3 hours after the injection, and concentration of the cholesterol component in the subject's kidney peaks at a time greater than 24 hours after the injection.

20. A method for administering a vaccination composition to a mammalian subject, comprising:
injecting the vaccination composition at an intramuscular injection site of the subject, the intramuscular injection site being associated with a draining lymph node (dLN);
wherein the vaccination composition comprises an antigen and a saponin-based adjuvant, the saponin-based adjuvant comprising a saponin component and a cholesterol component; and
wherein, following injection of the vaccination composition at the intramuscular injection site of the subject, concentration of the saponin component in one of the subject's intestines, lungs and heart remains approximately zero up to 168 hours after the injection, and concentration of the cholesterol component in the one of the subject's intestines, lungs and heart peaks at a time greater than 24 hours after the injection.

21. The method of embodiment 20, wherein the saponin-based adjuvant comprises nanoparticles of Q. saponaria saponin fraction A, cholesterol and phospholipid and nanoparticles of Q. saponaria saponin fraction C, cholesterol and phospholipid.

22. The method of any of embodiments 20-21, wherein the saponin-based adjuvant is mixed in a carrier.

23. A method for administering a vaccination composition to a mammalian subject, comprising:
injecting the vaccination composition at an intramuscular injection site of the subject, the intramuscular injection site being associated with a draining lymph node (dLN), the vaccination composition comprising a saponin-based adjuvant;
wherein a cytokine response induced by the vaccine composition at the dLN measured 24 hours after injection of the mixture is stronger than a cytokine response at the dLN measured 6 hours after injection of the mixture.

24. The method of embodiment 23, wherein the cytokine response is related to the abundance of at least one of the following cytokines: CXCL10, IL-1b, TNF-α, IL-30, IL-6, CXCL1, CCL2, IL-5, CCL3, IL-33, and IFN-g.

25. The method of any of embodiments 23-24, wherein abundance of at least two of the cytokines selected from the group consisting of CXCL10, IL-1b, TNF-α, IL-30, IL-6, CXCL1, CCL2, IL-5, CCL3, IL-33, and IFN-g at the dLN is greater at about 24 hours after the injection compared to 6 hours after the injection.

26. The method of any of embodiments 23-25, wherein abundance of at least three of the cytokines selected from the group consisting of CXCL10, IL-1b, TNF-α, IL-30, IL-6, CXCL1, CCL2, IL-5, CCL3, IL-33, and IFN-g at the dLN is greater at about 24 hours after the injection compared to 6 hours after the injection.

27. The method of any of embodiments 23-26, wherein the saponin-based adjuvant comprises nanoparticles of Q. saponaria saponin fraction A, cholesterol and phospholipid and nanoparticles of Q. saponaria saponin fraction C, cholesterol and phospholipid.

28. The method of any of embodiments 23-27, wherein the mammalian subject is a human.

29 The method of any of embodiments 23-28, wherein the saponin-based adjuvant is mixed with an antigen.

30. The method of any of embodiments 23-29, wherein the saponin-based adjuvant is mixed with a carrier.

1. A method of providing a vaccination composition to a mammalian subject, comprising:
injecting the vaccination composition at an intramuscular injection site of the subject, the vaccination composition comprising a saponin-based adjuvant and the intramuscular injection site being associated with a draining lymph node (dLN);
wherein, following injection of the vaccination composition at the intramuscular injection site, a fraction of T cells in the dLN that are positive for i) CD45 and ii) the saponin-based adjuvant has a minimum value at a time greater than one hour following injection of the vaccination composition at the intramuscular injection site and less than 24 hours following injection of the vaccination composition at the intramuscular injection site, the fraction of T cells in the dLN that are positive for i) CD45 and ii) the saponin-based adjuvant at a time 24 hours following injection of the vaccination composition having a value greater than the minimum value.

2. The method of claim 1, wherein the minimum value is more than about one tenth of a maximum value of the fraction of T cells in the dLN that are positive for i) CD45 and ii) the saponin-based adjuvant between one hour and twenty four hours following injection of the vaccination composition at the intramuscular injection site.

3. The method of claim 1, wherein the minimum value is more than about one eighth of a maximum value of the fraction of T cells in the dLN that are positive for i) CD45 and ii) the saponin-based adjuvant between one hour and twenty four hours following injection of the vaccination composition at the intramuscular injection site.

4. The method of claim 1, wherein the minimum value is more than about one fifth of a maximum value of the fraction of T cells in the dLN that are positive for i) CD45 and ii) the saponin-based adjuvant between one hour and twenty four hours following injection of the vaccination composition at the intramuscular injection site.

5. The method of claim 1, wherein the saponin-based adjuvant comprises nanoparticles of Q. saponaria saponin fraction A, cholesterol and phospholipid and nanoparticles of Q. saponaria saponin fraction C, cholesterol and phospholipid.

6. The method of claim 1, wherein the mammalian subject is a human.

7. The method of claim 1, wherein the saponin-based adjuvant is mixed with an antigen.

8. The method of claim 1, wherein the saponin-based adjuvant is mixed with a carrier.

9. A method of providing a vaccination composition to a mammalian subject, comprising:
injecting the vaccination composition at an intramuscular injection site of the subject, the intramuscular injection site being associated with a draining lymph node (dLN), the vaccination composition including a saponin-based adjuvant;
wherein, over a time period between about one hour and about twenty four hours following injection of the vaccination composition, a fraction of B cells in the dLN that are positive for i) CD45 and ii) the adjuvant has a maximum value at a time less than three hours following injection of the mixture at the intramuscular injection site.

10. The method of claim 9, wherein a value of the fraction of B cells in the downstream lymph node positive for i) CD45 and ii) the adjuvant that are B cells at about twenty four hours following injection of the mixture at the intramuscular injection site is less than about one tenth of the maximum value.

11. The method of claim 9, wherein a value of the fraction of cells in the downstream lymph node positive for i) CD45 and ii) the adjuvant that are B cells at about twenty four hours following injection of the mixture at the intramuscular injection site is less than about one twentieth of the maximum value.

12. The method of claim 9, wherein the saponin-based adjuvant comprises nanoparticles of Q. saponaria saponin fraction A, cholesterol and phospholipid and nanoparticles of Q. saponaria saponin fraction C, cholesterol and phospholipid.

13. The method of claim 9, wherein the mammalian subject is a human.

14. The method of claim 9, wherein the saponin-based adjuvant is mixed with an antigen.

15. The method of claim 9, wherein the saponin-based adjuvant is mixed with a carrier.

16. A method for administering a vaccination composition to a mammalian subject, comprising:
injecting the vaccination composition at an intramuscular injection site of the subject, the intramuscular injection site being associated with a draining lymph node (dLN);
wherein the vaccination composition comprises an antigen and a saponin-based adjuvant, the saponin-based adjuvant comprising a saponin component and a cholesterol component; and
wherein, following injection of the vaccination composition at the intramuscular injection site of the subject, concentration of the saponin component in the subject's blood plasma peaks at a time less than 3 hours after the injection, and concentration of the cholesterol component in the subject's blood plasma peaks at a time greater than 6 hours after the injection.

17. The method of claim 16, wherein the saponin-based adjuvant comprises nanoparticles of Q. saponaria saponin fraction A, cholesterol and phospholipid and nanoparticles of Q. saponaria saponin fraction C, cholesterol and phospholipid.

18. The method of claim 16, wherein the saponin-based adjuvant is mixed in a carrier.

19. The method of claim 16, wherein, following injection of the vaccination composition at the intramuscular injection site of the subject, concentration of the saponin component in the subject's kidney peaks at a time less than 3 hours after the injection, and concentration of the cholesterol component in the subject's kidney peaks at a time greater than 24 hours after the injection.

20-30. (canceled)
